(19) United States
(12) Patent Application Publication
(10) Pub. No.: US 2018/0185103 A1
(43) Pub. Date: Jul. 5, 2018

(54) MEDICAL TREATMENT ASSISTING APPARATUS

(71) Applicant: DENSO CORPORATION, Kariya-city, Aichi-pref. (JP)

(72) Inventors: Go MUKUMOTO, Kariya-city (JP); Tsuyoshi UEYAMA, Kariya-city (JP); Kazutaka TOYODA, Kariya-city (JP)

(21) Appl. No.: 15/740,120

(22) PCT Filed: Jun. 9, 2016
(86) PCT No.: PCT/JP2016/002791

§ 371 Date: Dec. 27, 2017

(30) Foreign Application Priority Data
Jul. 21, 2015 (JP) 2015-144037

(51) Int. Cl. A61B 34/30; A61C 1/08; A61C 8/00; A61C 19/04; A61B 90/00; B25J 9/16

(52) U.S. Cl. CPC A61B 34/30; A61C 1/084; A61C 8/0089; A61C 19/04; A61B 90/03; B25J 9/1676; Y10S 901/49; A61B 90/37; A61B 2034/306; Y10S 901/09; Y10S 901/41; Y10S 901/47; B25J 9/1697

(57) ABSTRACT

A medical treatment assisting apparatus includes a multi-joint robot, a position specification unit, an abnormality determination unit, and a control unit. The multi-joint robot has a multi-joint arm in which a plurality of arms are connected through a joint. The position specification unit specifies a treatment position that is the position of a portion of a patient to be subjected to a medical treatment in a real space. The abnormality determination unit determines whether or not a state of the treatment position based on the displacement of the treatment position specified by the position specification unit is in an abnormal state, which is predefined to indicate an abnormality. The control unit performs a safety control which increases the safety of the medical treatment when a determination result of the abnormality determination unit indicates that the state of the treatment position is in the abnormal state.

### CROSS REFERENCE TO RELATED APPLICATION

This application is based on Japanese Patent Application No. 2015-144037 filed on Jul. 21, 2015, the disclosure of which is incorporated herein by reference.

### TECHNICAL FIELD

The present disclosure relates to a medical treatment assisting apparatus which assists a medical treatment.

### BACKGROUND ART

Conventionally, medical treatment assisting apparatuses which assist medical treatments have been known (see, e.g., Patent Literature 1).

Medical treatment assisting apparatuses of this type include a medical treatment assisting apparatus which assists dental implantation as one of medical treatments. This medical treatment assisting apparatus includes a three-dimensional measuring instrument which specifies a treatment position as the portion of a patient into which an implant body is to be inserted, a drill unit having a drill bit to be used for a dental treatment, and a multi-joint robot which moves the tip of the drill bit to the treatment position. The three-dimensional measuring instrument in Patent Literature 1 is an image capturing device which captures a visible image.

### PRIOR ART LITERATURE

### Patent Literature

Patent Literature 1: JP 2013-236749 A

### SUMMARY OF INVENTION

In the medical treatment assisting apparatus described in Patent Literature 1, the treatment position is specified on the basis of the visible image captured using the three-dimensional measuring instrument.

When dental implantation is performed, the arm of the multi-joint robot, the body of a treatment provider, or the like may be interposed between the three-dimensional measuring instrument and the treatment position. As a result, the treatment position does not show up in the image captured using the three-dimensional measuring instrument, and thus may not be able to be precisely specified.

In such an abnormal state where the treatment position cannot be precisely specified, when the multi-joint robot is operated to move the drill bit, the drill bit is moved to a position unintended by the treatment provider and it may be difficult to ensure safety.

In short, the medical treatment assisting apparatus described in Patent Literature 1 is required to ensure safety in the medical treatment when the state of the treatment position is the abnormal state.

An object of the present disclosure is to provide a medical treatment assisting apparatus which can ensure safety in a medical treatment when a state of a treatment position is an abnormal state.

According to a first aspect of the present disclosure, a medical treatment assisting apparatus includes a multi-joint robot, a position specification unit, an abnormality determination unit, and a control unit.

The multi-joint robot has a multi-joint arm in which a plurality of arms are connected through a joint.

The position specification unit specifies a treatment position indicating the position of a portion of a patient to be subjected to a medical treatment in a real space. The abnormality determination unit determines whether or not a state of the treatment position based on the displacement of the treatment position specified by the position specification unit is in an abnormal state, which is predefined to indicate an abnormality.

The control unit performs a safety control which increases the safety of the medical treatment when a result of the determination by the abnormality determination unit indicates that the state of the treatment position is in the abnormal state.

Such a medical treatment assisting apparatus allows the safety control to be performed when the state of the treatment position is in the abnormal state. The safety control is control which increases the safety of the medical treatment.

Accordingly, even in the abnormal state, the medical treatment assisting apparatus can ensure safety in the medical treatment.

According to a second aspect of the present disclosure, the medical treatment assisting apparatus may also include a range setting unit. The range setting unit sets a position movable range indicating a range in a space in which the treatment position may possibly move.

In this case, the abnormality determination unit assumes that the state of the treatment position is the treatment position specified by the position specification unit. When the treatment position is outside the position movable range set by the range setting unit, the abnormality determination unit may determine that the state of the treatment position is in the abnormal state.

In such a medical treatment assisting apparatus, it is possible to determine that the state of the treatment position is in the abnormal state when the treatment position is outside the position movable range.

### BRIEF DESCRIPTION OF DRAWINGS

The above and other objects, features and advantages of the present disclosure will become more apparent from the following detailed description made with reference to the accompanying drawings, in which:

FIG. 1 is an illustrative view illustrating a schematic configuration of a medical treatment assisting apparatus;

FIG. 2 is a block diagram illustrating a control system in the medical treatment assisting apparatus;

FIG. 3 is an illustrative view showing an example of a position tracking device;

FIG. 4 is an illustrative view showing an example of the position where the medical treatment assisting apparatus is placed;

FIG. 5 is a flow chart illustrating the procedure of a medical treatment assisting process;

FIG. 6A is a view obtained by viewing the movable range of a treatment position from above;

FIG. 6B is a view showing the movable range of the treatment position in an X-Z plane; and

FIG. 6C is a view showing the movable range of the treatment position along an X-axis.

### DESCRIPTION OF EMBODIMENTS

Embodiments of the present disclosure will be described hereinafter with reference to the drawings.

<Medical Treatment Assisting System>

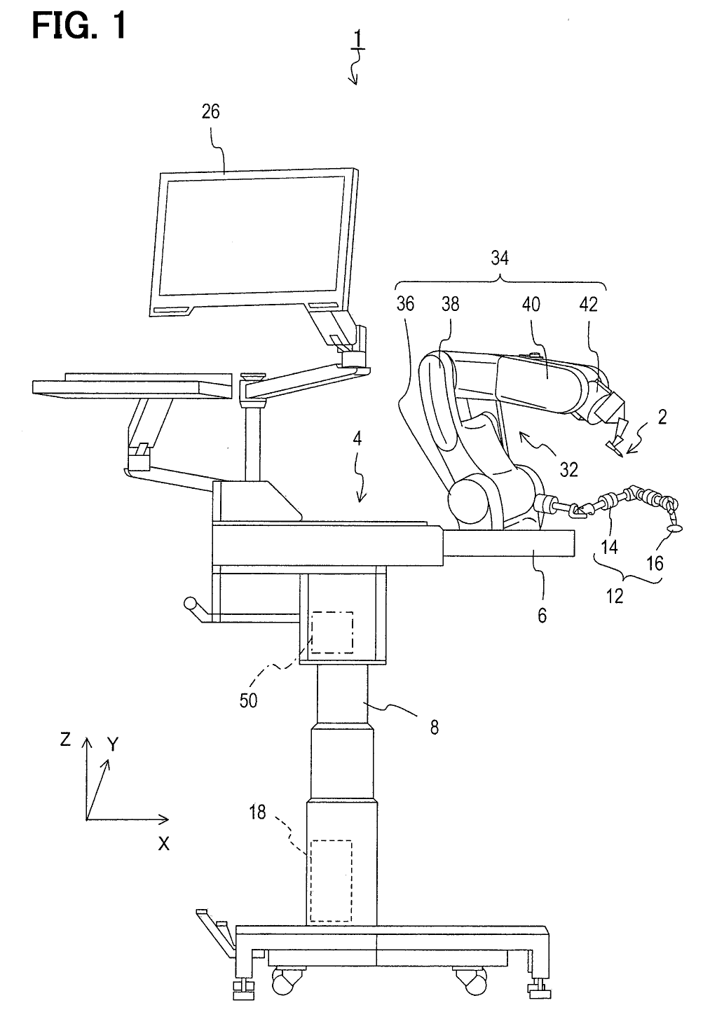

A medical treatment assisting system 1 shown in FIG. 1 is the system which assists a medical treatment. The medical treatment assisting system 1 in the present embodiment is an example of a medical treatment assisting apparatus.

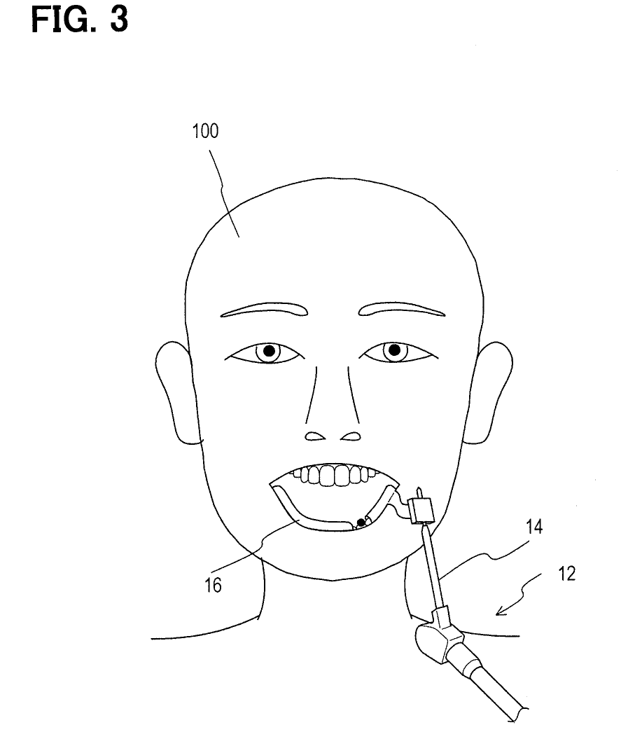

In the present embodiment, as the medical treatment, dental implantation is assumed. The dental implantation is a dental surgery which inserts an implant body into the jawbone of a patient 100 (see FIG. 3) and attaches a prosthetic material to the inserted implant body. The position in (portion of) the jawbone of the patient 100 into which the implant body is to be inserted is hereinafter referred to as a treatment position.

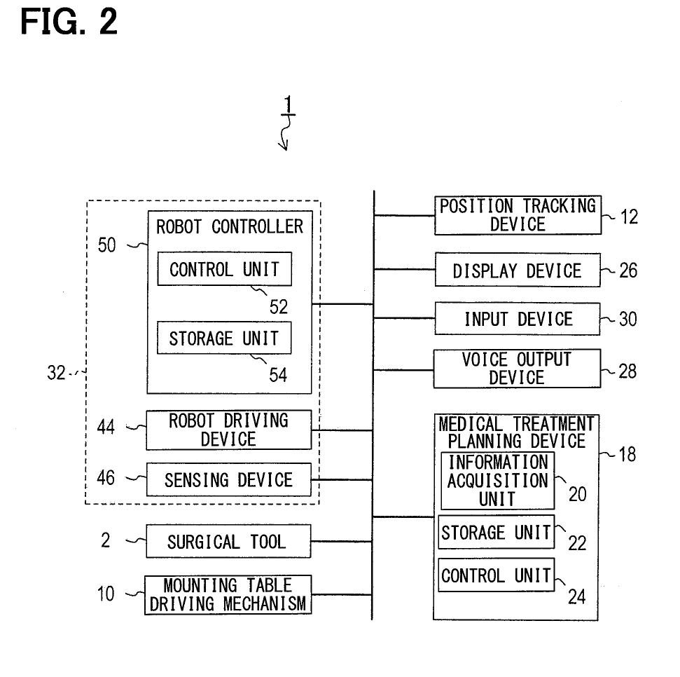

As shown in FIGS. 1 and 2, the medical treatment assisting system 1 includes a surgical tool 2, a mounting table 4, a position tracking device 12, a medical treatment planning device 18, a display device 26, a voice output device 28, an input device 30, and a guide robot 32.

The surgical tool 2 is an instrument used for a medical treatment. The surgical tool 2 is attached to the tip of the guide robot 32.

The surgical tool 2 in the present embodiment includes a drill unit used for a dental treatment. The drill unit has various drill bits used for a dental treatment and a driving mechanism which drives the drill bits. Note that the drill unit mentioned herein includes so-called dental handpieces. The dental handpieces include a so-called straight-geared-angle handpiece and a so-called contra handpiece.

The medical treatment assisting system 1 assists dental implantation performed by a treatment provider using the surgical tool 2 attached to the tip of the guide robot 32. The “assisting of dental implantation” mentioned herein includes the movement of the surgical tool 2 to the treatment position as the portion of the patient 100 into which the implant body is to be inserted. The movement of the surgical tool 2 includes the entering of the surgical tool 2 into the mouth of the patient 100 and the movement of the surgical tool 2 to a changing position where the surgical tool 2 is changed.

The “assisting of dental implantation” mentioned herein may also include drilling a hole in the jawbone using the drill as the surgical tool 2.

The mounting table 4 is a mechanism on which the guide robot 32 is placed. The mounting table 4 includes a top panel 6, a supporting portion 8, and a mounting table driving mechanism 10 (see FIG. 2).

The top panel 6 is a plate-like member to which the guide robot 32 is fixed. The supporting portion 8 is a support horizontally supporting the top panel 6. The mounting table driving mechanism 10 has a motor which drives the top panel 6 along each of an X-axis direction, a Y-axis direction, and a Z-axis direction.

That is, the mounting table 4 is configured so as to move the guide robot 32 placed on the top panel 6 along a horizontal plane and a vertical direction as intended.

The position tracking device 12 is a device which specifies the relative positional relationship between a reference point defined by the guide robot 32 and the treatment position.

The position tracking device 12 in the present embodiment has an arm 14 as a multi-joint arm extending from an arm reference point and an attachment 16 attached to the tip of the arm 14. The arm reference point mentioned herein is a position in the guide robot 32 where the extension of the arm 14 is initiated.

The attachment 16 is attached to the affected portion of the patient 100. As the attachment 16 in the present embodiment, as shown in FIG. 3, a mouthpiece in which the treatment position is marked is used.

That is, the position tracking device 12 is a known tracking arm which specifies the marked position in the attachment 16 as the treatment position on the basis of a relative position with respect to the arm reference point.

The medical treatment planning device 18 specifies the treatment position and makes a plan for a medical treatment to be performed on the specified treatment position. The medical treatment planning device 18 includes an information acquisition unit 20, a storage unit 22, and a control unit 24. The information acquisition unit 20 acquires information (hereinafter referred to as “position specification information”) required to specify the treatment position.

The information acquisition unit 20 in the present embodiment acquires a plurality of tomographic images produced by a computed tomographic (CT) device as the position specification information.

The storage unit 22 is a known storage device which stores data and a processing program.

The control unit 24 is a known control device having a known microcomputer including at least a ROM, a RAM, and a CPU and executes processing in accordance with the processing program stored in the storage unit 22.

Note that, in the storage unit 22 of the medical treatment planning device 18, the processing program for the control unit 24 to execute a medical treatment plan producing process for producing a “medical treatment plan” is stored.

In the medical treatment plan producing process, the “medical treatment plan” is produced in accordance with the three-dimensional coordinate information of the jawbone of the patient 100 based on the position specification information. The production of the “medical treatment plan” includes performing the specification of the treatment position where an implant body is to be inserted, the specification of an angle at which the implant body is to be inserted, and the specification of a depth to which the implant body is to be inserted in a planning coordinate system. Note that the treatment position includes information about whether the jawbone into which the implant body is to be inserted is an upper jawbone or a lower jawbone and about the position of a tooth in the jawbone. The “medical treatment plan” may also include the timing of entering the surgical tool 2 into the mouth of the patent 100 during the operation of the guide robot 32 when the medical treatment is performed at the treatment position and the angle of entrance of the surgical tool 2 into the mouth of the patient 100.

Note that the planning coordinate system mentioned herein is a coordinate system (e.g., a coordinate system in a CT image) in the “medical treatment plan” produced in the medical treatment planning device 18.

The “medical treatment plan” will be hereinafter referred to as required information for medical treatment. Note that, since a method for producing the “medical treatment plan” is known, a detailed description thereof is omitted herein.

The display device 26 is a known device (e.g., a liquid crystal display) which displays an image. The voice output device 28 is a known device (e.g., a speaker) which outputs a voice.

The input device 30 is a known device which receives an input of information. The input device 30 includes various input devices such as a keyboard, a pointing device, and a switch. The pointing device mentioned herein includes a known mechanism such as a touch pad or a touch panel.

<Guide Robot>

The guide robot 32 is a known vertical multi-joint robot including a multi-joint arm 34 and a robot controller 50.

The multi-joint arm 34 includes a base portion 36 fixed to the mounting table 4, an upper arm portion 38 and a front arm portion 40 which form an arm extending from the base portion 36, and a hand attachment portion 42 which is located at the tip of the front arm portion 40 and to which the surgical tool 2 is attached.

The base portion 36, the upper arm portion 38, the front arm portion 40, and the hand attachment portion 42 which are included in the multi-joint arm 34 are connected to each other via joint portions. Each of the joint portions includes a robot driving device 44 and a sensing device 46.

The robot driving device 44 is a device which drives the multi-joint arm 34. As the robot driving device 44, a motor which drives each of the joint portions is used. The sensing device 46 is intended to detect the coordinates of the tip (and the surgical tool 2) of the multi-joint arm 34. As the sensing device 46, e.g., a rotary encoder which detects the rotation angle of each of the robot driving devices 44 may also be used.

That is, the multi-joint arm 34 is a known arm having a plurality of movable portions in a three-dimensional coordinate system (X-, Y-, and Z-coordinate system) for real space. To the tip (i.e., hand attachment portion 42) of the multi-joint arm 34, the surgical tool 2 is attached.

The robot controller 50 drives the robot driving devices 44 of the guide robot 32 in accordance with the results of sensing by the sensing devices 46. The robot controller 50 includes a control unit 52 and a storage unit 54.

The control unit 52 is a known control device having a known microcomputer including at least a ROM, a RAM, and a CPU. The storage unit 54 is a known device which stores information and data.

The storage unit 54 stores a processing program for the robot controller 50 to execute a medical treatment assisting process in which the medical treatment performed by the treatment provider is assisted by the medical treatment assisting system 1.

<Medical Treatment Assisting Process>

Next, a description will be given of the medical treatment assisting process executed by the robot controller 50.

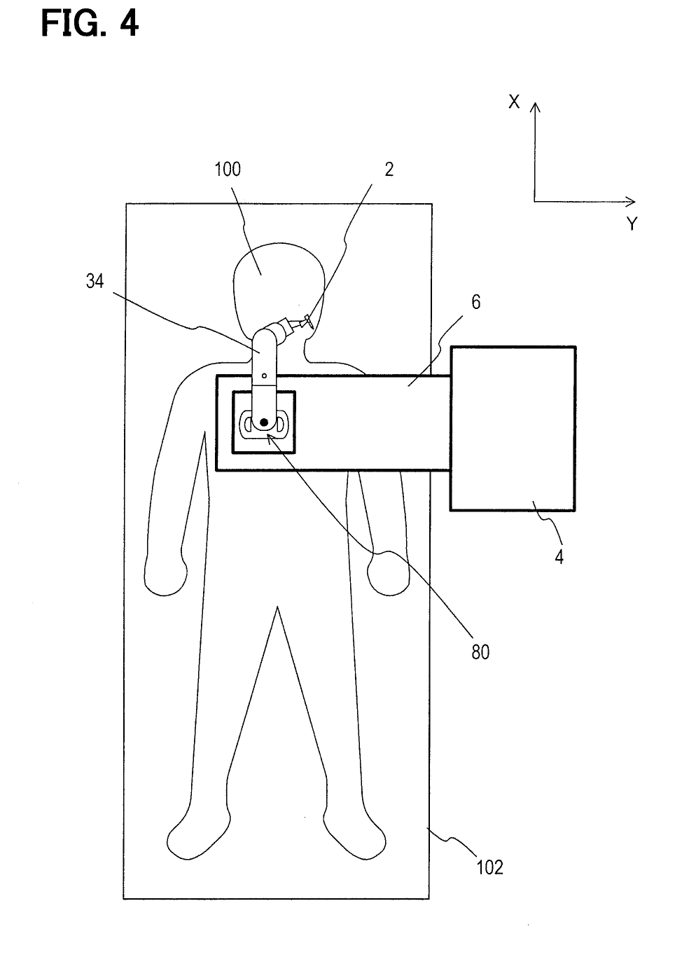

The medical treatment assisting process is activated after the medical treatment assisting system 1 is installed at a device installation place 80 specified in advance as shown in FIG. 4 and brought into a state where the treatment position of the patient 100 can be tracked using the position tracking device 12. Note that, as an example of the device installation place 80, a position where at least a part of the chest region of the patient 100 lying on a bed (surgical bed) 102 is covered with the top panel 6 of the mounting table 4.

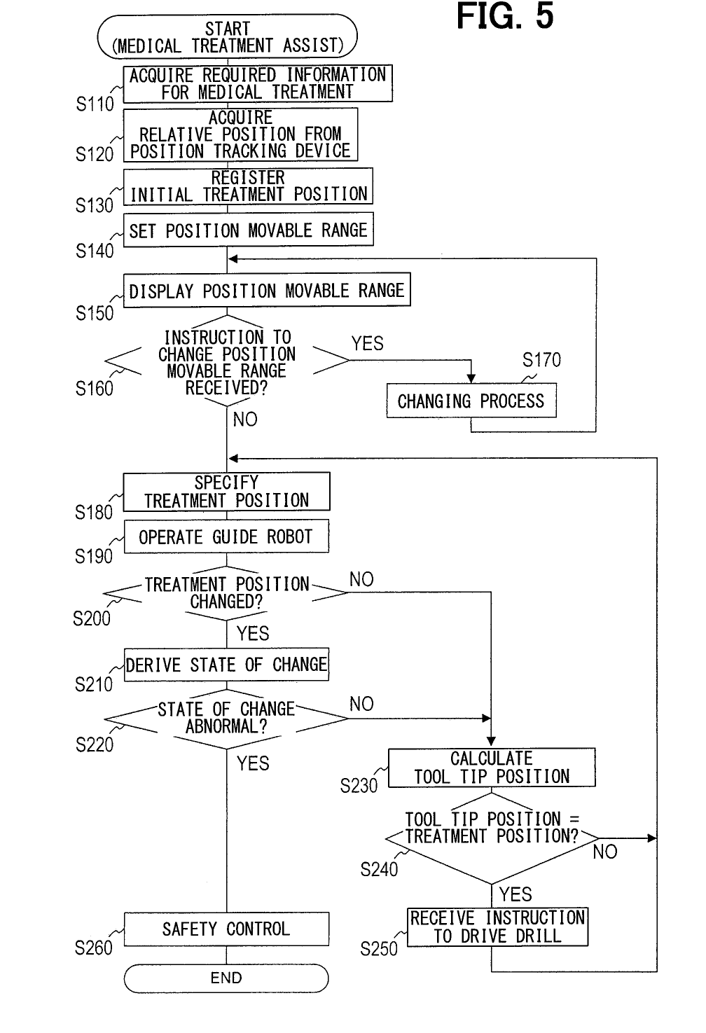

Then, when the medical treatment assisting process is activated, as shown in FIG. 5, the control unit 52 of the robot controller 50 acquires the required information for medical treatment from the medical treatment planning device 18 (S110).

Then, in the medical treatment assisting process, the control unit 52 acquires, from the position tracking device 12, the relative position of the marked portion of the attachment 16 of the position tracking device 12 with respect to the arm reference point (S120). Subsequently, in the medical treatment assisting process, the control unit 52 registers the initial position of the treatment position of the patient 100 in the three-dimensional coordinate system for real space on the basis of the relative position of the marked portion of the attachment 16 acquired in S120 (S130). The registration of the initial position of the treatment position in S130 may appropriately be performed in accordance with a known registration. The registration is a process which converts the treatment position in the planning coordinate system that has been specified by the medical treatment planning device 18 to the treatment position in the three-dimensional coordinate system for real space and registers the treatment position.

Specifically, in the registration, coordinates ^{TRAK}T_{CT} of the marked portion of the attachment 16 in the planning coordinate system is calculated in accordance with the following expression (1).

^{TRAK}T_{CT}=^{TRAK}T_{TRAKtip}·^{TRAKtip}T_{A′}·^{A′}T_{A}·^{CT}T⁻¹_{A} (1)

Note that the symbol “^{TRAK}T_{TRAKtip}” in the expression (1) shows the position of the tip of the arm 14 in the position tracking device 12 in the three-dimensional coordinate system for real space. Also, the symbol “^{TRAKtip}T_{A′}” shows the position of the attachment 16 in the position tracking device 12 in the three-dimensional coordinate system for real space. The symbol “^{A′}T_{A}” shows a value which corrects an attachment error associated with the attachment 16.

In the registration, in accordance with the following expression (2), the coordinates of the marked portion of the attachment 16 in the three-dimensional coordinate system for real space, i.e., the initial position ^{TRAK}T_{IMP} of the treatment position is calculated.

^{TRAK}T_{IMP}=^{TRAK}T_{CT}·^{CT}T_{IMP} (2)

In the expression (2), the symbol “^{CT}T_{IMP}” shows the treatment position in the planning coordinate system.

The registration allows the relative position of the marked portion of the attachment 16 with respect to the arm reference point that has been determined by the position tracking device 12 to be tracked as the treatment position in the three-dimensional coordinate system for real space.

Note that, in the present embodiment, a description is given on the assumption that calibration has been performed. The calibration mentioned herein is a process which places the relative positional relationship between a tool tip position and the treatment position in a shared coordinate system. The shared coordinate system mentioned herein is the three-dimensional system for real space which is, e.g., a robot coordinate system in the guide robot 32.

Also, the tool tip position mentioned herein is a tip position showing the tip portion of the surgical tool 2 which is, e.g., the position of the tip portion of a drill bit.

Subsequently, in the medical treatment assisting process, the control unit 52 sets a position movable range (S140). The position movable range mentioned herein means a range in a real space in which the treatment position may possibly move.

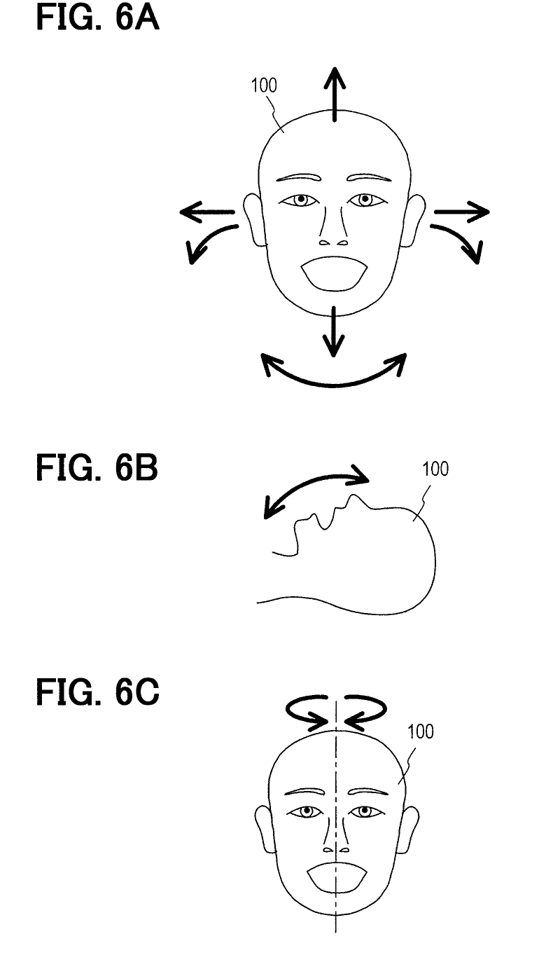

That is, as shown in FIGS. 6A, 6B, and 6C, even when the patient 100 is lying on the bed 102, the head portion of the patient 100 can move in front-rear/lateral/vertical directions. Since the treatment position in a real space moves with the movement of the head portion, a range fixed in a real space assumed to be a range in which the treatment position moves is set as the position movable range.

Then, in the medical treatment assisting process, the control unit 52 of the robot controller 50 outputs the position movable range set in S140 to the display device 26 (S150). The display device 26 displays the position movable range.

Then, in the medical treatment assisting treatment, the control unit 52 determines whether or not an instruction to change the position movable range is received via the input device 30 (S160). Note that the instruction to change the position movable range may be received by correcting the position movable range displayed on the display device 26 via the input device 30.

When the result of the determination in S160 indicates that the instruction to change the position movable range is received (S160: YES), the control unit 52 changes the position movable range in accordance with the received instruction to change (S170). Note that the changing of the position movable range mentioned herein is the changing of a position to which the position movable range is to be set or the updating of the size of the position movable range.

Then, the control unit 52 returns the medical treatment assisting process to S150. In S150, the control unit 52 causes the display device 26 to display the changed position movable range.

On the other hand, when the result of the determination in S160 indicates that the instruction to change the position movable range is not received (S160: NO), the control unit 52 moves the medical treatment assisting process to S180. In S180, the control unit 52 specifies the treatment position in the three-dimensional coordinate system for real space on the basis of the relative position of the marked portion of the attachment 16 with respect to the arm reference point that has been determined by the position tracking device 12. That is, in S180, which is repeatedly performed in the medical treatment assisting process, the treatment position is sequentially specified along a time axis.

Subsequently, in the medical treatment assisting process, the control unit 52 moves the guide robot 32 so as to move the surgical tool 2 to the treatment position in a real space (S190).

Also, in the medical treatment assisting process, the control unit 52 determines, on the basis of the treatment position sequentially specified along the time axis in S180, whether or not the treatment position has changed along the time axis (i.e., a shift of the treatment position) (S200). When the result of the determination in S200 indicates that the treatment position has not changed along the time axis (S200: NO), the control unit 52 moves the medical treatment assisting process to S230 described later in detail.

On the other hand, when the result of the determination in S200 indicates that the treatment position has changed along the time axis (S200: YES), the control unit 52 moves the medical treatment assisting process to S210. In S210, the control unit 52 derives a state of change that represents a state of change in the treatment position based on the displacement of the treatment position along the time axis.

The state of change mentioned herein means the state of change in the treatment position. The state of change includes, e.g., the very treatment position in a real space, a fluctuation in treatment position, and the acceleration rate of the treatment position.

Note that the fluctuation in treatment position is a variation of the treatment position from the average position thereof. The fluctuation in treatment position includes, for example, a mean-square fluctuation (i.e., variance). Note that fluctuations may appropriately be calculated using a known method.

The acceleration rate of the treatment position is the change rate of the speed of the attachment 16 showing the treatment position per unit time. Since a method for calculating the acceleration rate is known, a detailed description thereof is omitted herein.

Then, in the medical treatment assisting process, the control unit 52 determines whether or not the state of change is an abnormal state (S220).

The abnormal state mentioned herein is predefined as a state of the treatment position having an abnormality.

When it is assumed that the state of change is represented by the very treatment position in a real space, it is possible to consider that an example of the abnormal state is a state where the treatment position is present outside the position movable range. When it is assumed that the state of change is represented by the fluctuation in treatment position, it is possible to consider that an example of the abnormal state is provided by a first threshold that is predefined. The first threshold mentioned herein is defined in advance as a value showing that the fluctuation in treatment position is abnormal.

When it is assumed that the state of change is represented by the acceleration rate of the treatment position, it is possible to consider that an example of the abnormal state is provided by a second threshold predefined. The second threshold mentioned herein is defined in advance as a value showing that the acceleration rate of the treatment position is abnormal.

When the treatment position in a real space is present outside the position movable range in S220, it may also be possible to determine that the state of change is in the abnormal state. When the fluctuation in treatment position is equal to or more than the first threshold in S220, it may also be possible to determine that the state of change is in the abnormal state. When the acceleration rate of the treatment position is equal to or more than the second threshold in S220, it may also be possible to determine that the state of change is in the abnormal state.

When the result of the determination in S220 indicates that the state of change is in the abnormal state (S220: YES), the control unit 52 moves the medical treatment assisting process to S260 described later in detail. On the other hand, when the result of the determination in S220 indicates that the state of change is not in the abnormal state (S220: NO), the control unit 52 moves the medical treatment assisting process to S230.

Note that the medical treatment assisting process is moved also to S230 when the result of the determination in S200 indicates that the treatment position has not changed along the time axis (S200: NO).

In S230, the control unit 52 specifies the tool tip position. As a method for specifying the tool tip position, e.g., a known method which specifies the tool tip position on the basis of the relative positional relationship with the arm reference point of the multi-joint arm 34 can be considered.

Subsequently, the control unit 52 determines whether or not the tool tip position specified in S230 coincides with the treatment position (S240). When the result of the determination in S240 indicates that the tool tip position does not coincide with the treatment position (S240: NO), then, the control unit 52 returns the medical treatment assisting process to S180.

On the other hand, when the result of the determination in S240 indicates that the tool tip position coincides with the treatment position (S240: YES), the control unit 52 receives an input of a drill instruction as an instruction to drive the drill (S250). The control unit 52 to which the drill instruction has been input rotates the drill bit of the surgical tool 2. Then, the treatment provider drills a hole in the jawbone using the surgical tool 2.

Subsequently, the control unit 52 returns the medical treatment assisting process to S180.

In S260 to which the medical treatment assisting process is moved when the result of the determination in S200 indicates that the state of change is in the abnormal state, the control unit 52 performs a safety control which increases the safety of the medical treatment.

As an example of the safety control, halting the operation of the guide robot 32 so as to halt the movement of the tip portion of the multi-joint arm 34 and thus halt the movement of the surgical tool 2 can be considered.

As another example of the safety control, making a notification that the attachment 16 is present outside the movable range, i.e., that the attachment 16 is off the affected portion of the patient 100 can be considered. It can be considered that, to make the notification mentioned herein, the control unit 52 outputs a signal showing the presence of the attachment 16 outside the movable range to the display device 26 and to the voice output device 28. In this case, the display device 26 that has acquired the signal may appropriately display the presence of the attachment 16 outside the movable range and the voice output device 28 that has acquired the signal may appropriately produce a voice output announcing the presence of the attachment 16 outside the movable range.

Subsequently, the control unit 52 ends the current medical treatment assisting process.

[Effects of Embodiment]

As has been described hereinabove, the medical treatment assisting process allows the safety control to be performed when the state of change is in the abnormal state. The safety control is control which increases the safety of a medical treatment.

As a result, the medical treatment assisting process can ensure safety in a medical treatment even when the change of the treatment position is in the abnormal state.

In the medical treatment assisting process, it is notified that the attachment 16 is present outside the movable range. This allows the user of the medical treatment assisting system 1 to recognize that the state of the treatment position is in the abnormal state.

In particular, in the medical treatment assisting process, the movement of the tip portion of the multi-joint arm 34 is halted as the safety control. Accordingly, even when the state of change is in the abnormal state, the medical treatment assisting process can more reliably ensure safety in a medical treatment.

Also, the medical treatment assisting process assumes that a condition to determine that the state of change is in the abnormal state is that the treatment position is outside the movable range. As a result, in the medical treatment assisting process, when the attachment 16 of the position tracking device 12 comes off the affected portion of the patient 100, the safety control can be performed on the assumption that the treatment position is outside the position movable range. This can further increase safety in a medical treatment.

Also, the medical treatment assisting process assumes that a condition to determine that the state of change is in the abnormal state is when the fluctuation in treatment position is equal to or more than the first threshold or when the acceleration rate of the treatment position is equal to or more than the second threshold. Therefore, when the body movement of the patient 100 is large, the medical treatment assisting process allows the safety control to be performed on the assumption that the treatment position is outside the position movable range. This can further increase safety in a medical treatment.

Note that, in the medical treatment assisting process, the user of the medical treatment assisting system 1 can adjust the movable range. This allows the user of the medical treatment assisting system 1 to set the movable range which is convenient in performing a medical treatment and more smoothly perform the medical treatment.

[Other Embodiments]

While the embodiment of the present disclosure has been described hereinabove, the present disclosure is not limited to the embodiment described hereinabove. The present disclosure can be implemented in various other ways within the scope not departing from the gist of the present disclosure.

In the above-mentioned embodiment, the position tracking device 12 is formed of a known tracking arm, but the position tracking device 12 is not limited thereto. That is, the position tracking device 12 may be a device which emits a probing wave such as an infrared beam having an increased directionality to track a change in treatment position or may also be a device capable of specifying the treatment position in accordance with another method. In the former case, a mechanism which emits the probing wave may be placed over a space in which the medical treatment assisting system 1 is disposed or may be placed on the affected portion of the patient 100.

In other words, the position tracking device 12 may be any device as long as the device can specify the relative positional relationship between the reference point defined by the guide robot 32 and the treatment position.

In the above-mentioned embodiment, the information acquisition unit 20 of the medical treatment planning device 18 acquires the position specification information from the computer tomographic device. However, the information acquisition unit 20 of the medical treatment planning device 18 may acquire the position specification information from another device.

In the above-mentioned embodiment, as the medical treatment assisted by the medical treatment assisting system 1, dental implantation is assumed. However, the medical treatment assisted by the medical treatment assisting system 1 is not limited to dental implantation. For example, the medical treatment assisted by the medical treatment assisting system may be a surgical operation, an internal medicine treatment, a dental treatment other than dental implantation, or another medical treatment.

The treatment position is not limited to the position in (portion of) the jawbone of a patient in which an implant body is to be inserted. For example, the treatment position may be the portion of the patient 100 who needs a surgical operation, the portion of the patient 100 who needs an internal medicine treatment, or the portion of the patient 100 who needs a dental treatment.

Note that, in such a case, the surgical tool 2 attached to the tip of the guide robot 32 is not limited to the drill unit used for a dental treatment and may be an instrument used for various medical treatments. The instrument used for various medical treatments in this case may be a surgical instrument such as a scalpel or forceps and may be another medical instrument.

Alternatively, the medical treatment assisting system 1 may also include a stop switch for stopping the guide robot 32 from assisting a medical treatment or a switch for receiving the result of determining whether or not a medical treatment is to be assisted by the guide robot 32.

It is noted that a flowchart or the processing of the flowchart in the present disclosure includes sections (also referred to as steps), each of which is represented, for instance, as S100. Further, each section can be divided into several sub-sections while several sections can be combined into a single section. Furthermore, each of thus configured sections can be also referred to as a circuit, device, module, or means.

While the present disclosure has been described with reference to embodiments thereof, it is to be understood that the disclosure is not limited to the embodiments and constructions. The present disclosure is intended to cover various modification and equivalent arrangements. In addition, while the various combinations and configurations, other combinations and configurations, including more, less or only a single element, are also within the spirit and scope of the present disclosure.

What is claimed is:

1. A medical treatment assisting apparatus comprising:
a multi-joint robot that has a multi-joint arm including a plurality of arms connected by a joint;
a position specification unit that specifies a treatment position which is a position of a portion of a patient to be subjected to a medical treatment in a real space;
an abnormality determination unit that determines whether or not a state of the treatment position based on a displacement of the treatment position specified by the position specification unit is in an abnormal state, the abnormal state being predefined to indicate an abnormality;
a control unit that performs a safety control to increase safety of the medical treatment when a determination result of the abnormality determination unit indicates that the state of the treatment position is in the abnormal state; and
a range setting unit that sets a position movable range which is a range in a space in which the treatment position may possibly move, wherein
the abnormality determination unit assumes that the state of the treatment position is represented by the treatment position specified by the position specification unit, and
the abnormality determination unit determines that the state of the treatment position is in the abnormal state when the treatment position specified is outside the position movable range set by the range setting unit.

2. (canceled)

3. The medical treatment assisting apparatus according to claim 1, further comprising:
a reception unit that receives a change in the position movable range set by the range setting unit; and
an updating unit that updates the position movable range set by the range setting unit in accordance with the change in the position movable range which is received by the reception unit, wherein
the range setting unit sets the position movable range updated by the updating unit as a new position movable range.

4. A medical treatment assisting apparatus, comprising:
a multi-joint robot that has a multi-joint arm including a plurality of arms connected by a joint;
a position specification unit that specifies a treatment position which is a position of a portion of a patient to be subjected to a medical treatment in a real space;
an abnormality determination unit that determines whether or not a state of the treatment position based on a displacement of the treatment position specified by the position specification unit is in an abnormal state, the abnormal state being predefined to indicate an abnormality; and
a control unit that performs a safety control to increase safety of the medical treatment when a determination result of the abnormality determination unit indicates that the state of the treatment position is in the abnormal state, wherein
the position specification unit repeatedly specifies the treatment position along a time axis, and
the determination unit specifies a fluctuation in the treatment position as the state of the treatment position in accordance with shifting of the treatment position specified by the position specification unit, and determines that the state of the treatment position is in the abnormal state when the fluctuation in the treatment position specified is equal to or more than a first threshold that is predefined.

5. A medical treatment assisting apparatus, comprising:
a multi-joint robot that has a multi-joint arm including a plurality of arms connected by a joint;
a position specification unit that specifies a treatment position which is a position of a portion of a patient to be subjected to a medical treatment in a real space;
an abnormality determination unit that determines whether or not a state of the treatment position based on a displacement of the treatment position specified by the position specification unit is in an abnormal state, the abnormal state being predefined to indicate an abnormality; and
a control unit that performs a safety control to increase safety of the medical treatment when a determination result of the abnormality determination unit indicates that the state of the treatment position is in the abnormal state, wherein
the position specification unit repeatedly specifies the treatment position along the time axis, and
the determination unit specifies an acceleration rate of the treatment position as the state of the treatment position in accordance with shifting of the treatment position specified by the position specification unit, and determines that the state of the treatment position is in the abnormal state when the acceleration rate of the treatment position specified is equal to or more than a second threshold that is predefined.

6. The medical treatment assisting apparatus according to claim 1, wherein
the control unit makes a notification that the state of the treatment position is in the abnormal state as the safety control.

7. The medical treatment assisting apparatus according to claim 1, wherein
the control unit halts movement of a tip portion of the multi-joint arm as the safety control.
